(19) United States
(12) Patent Application Publication
(10) Pub. No.: US 2019/0314327 A1
(43) Pub. Date: Oct. 17, 2019

(54) Pharmaceutical Compositions Containing Cannabis, Uses Thereof and Methods for Improving Digestion and/or Treating Symptoms Associated with Gastrointestinal Complications

(71) Applicant: Exzell Pharma Inc., Markham (CA)

(72) Inventor: Pardeep Nijhawan, Markham (CA)

(21) Appl. No.: 16/375,919

(22) Filed: Apr. 5, 2019

(30) Foreign Application Priority Data
Apr. 11, 2018 (CA) 3000981

(51) Int. Cl. A61K 31/352; A61P 1/14; A61K 31/05; A61K 36/185; A61K 38/47

(52) U.S. Cl. CPC A61K 31/352; A61P 1/14; C12Y 302/01003; A61K 36/185; A61K 38/47; A61K 31/05

(57) ABSTRACT

A pharmaceutical composition comprising cannabis for improving digestion and/or treating the symptoms associated with gastrointestinal complications in a patient. Also provided is a use of the pharmaceutical composition for improving digestion and/or treating the symptoms associated with gastrointestinal complications in a patient, and a method for improving digestion and/or treating the symptoms associated with gastrointestinal complications. The pharmaceutical composition preferably includes a combination of therapeutically effective amount of one or more of the following medicinal ingredients: cannabis and/or an enzyme.

### FIELD OF THE INVENTION

The present invention relates to pharmaceutical compositions containing cannabis and methods for improving digestion and/or treating symptoms associated with gastrointestinal complications.

### BACKGROUND OF THE INVENTION

Gastrointestinal symptoms are very common in North America. In one study, millions of Americans in one year sought help for a number of gastrointestinal (GI) symptoms. 27 million patients sought help for abdominal pain, 5.6 million patients sought help for diarrhea, 11.1 million patients sought help for nausea and vomiting and another 18 million patients for a variety of GI conditions. (See: Perry A, Crockett S. Burden of Gastrointestinal, Liver and Pancreatic Diseases in the United States. Gastroenterology, 2015 December; 149(7):1731-1741)

As common as these symptoms are, the number of treatment options are extensive, yet many patients still do not experience complete resolution. This is more common with nausea, bloating, indigestion and stomach aches and cramps. The medications used for abdominal pain and cramps are mostly antidepressants that may be associated with significant side effects especially when used long term. The four classes of medication used are Selective Serotonin Reuptake Inhibitors (SSRIs), Serotonin-Norephinephrine Reuptake Inhibitors (SNRIs), Benzodiazepines, and Tricyclic Antidepressants (TCA). SSRIs are known to cause insomnia, sexual dysfunction, peptic ulcer disease and weight gain. SNRIs can cause stomach upset, insomnia, headaches, sexual dysfunction and increase in blood pressure. Benzodiazepines can cause drowsiness and can lead to dependency. TCAs also have significant side effects including orthostatic hypotension, constipation, urinary retention, dry mouth and blurry vision.

It has been reported that cannabis is known to help relieve nausea, anorexia, abdominal pain, diarrhea, irritable bowel syndrome and help in Crohns disease (See: Malik S, Baik D, Schey R. The role of cannabinoids in regulation of nausea and vomiting, and visceral pain. Curr Gastroenterolo Rep, 2015 February; 17(2):429 and Goyal H, Singla U, Gupta U, May E. Role of cannabis in digestive disorders. Eur J Gastroenterol Hepatol 2017 February; 29(2);135-143). Moreover, it may have also been demonstrated that higher levels of THC (tetrahydrocannabinol) may be desired to binds the CB (cannabinoid) receptors in order to help ameliorate effects in bowel disease. (See: Schicho R, Storr M. Cannabis finds its way into treatment of Crohn's disease. Pharmacology, 2014: 93(0):1-3.). Chronic use of cannabis, however, can lead to cannabis-induced nausea and suppressed appetite. Patients who use cannabis for longer than three months are considered chronic users. These patients are more likely to develop the GI complications associated with chronic cannabis use including anorexia, nausea, abdominal pain and change in bowel movements.

What may be needed is a pharmaceutical composition of cannabis that improves digestion and/or prevents a reduction in digestive function while minimizing occurrences and/or severity of side effects or gastrointestinal complications that may be associated with chronic use of cannabis.

Moreover, it would be advantageous to have a pharmaceutical composition of cannabis that improves digestion and/or prevents a reduction in digestive function while treating symptoms associated with gastrointestinal complications.

### SUMMARY OF THE INVENTION

According to an aspect of the present invention, there is disclosed a pharmaceutical composition for improving digestion and/or treating symptoms associated with gastrointestinal complications. The pharmaceutical composition may preferably, but need not necessarily, comprise, alone or in combination, therapeutically effective amounts of cannabis and/or enzymes for use as a medicinal product prescribed by a physician, a health care practitioner or an over-the-counter product available at pharmacies, marijuana dispensaries and/or mass food stores.

According to an aspect of the invention, there is preferably disclosed a pharmaceutical composition for improving digestion and/or treating symptoms associated with gastrointestinal complications including bloating, nausea, diarrhea, abdominal cramps, symptoms associated with Irritable Bowel Syndrome and Inflammatory Bowel Disease. The pharmaceutical composition may preferably, but need not necessarily, additionally comprise therapeutically effective amounts of digestive enzymes 1,4-alpha-D-Glucan glucohydrolase, 4-alpha-D-Glucan glucanohydrolase, Beta-D-galactoside galactohydrolase, Malt diastase, Protease, Triacylglycerol lipase, Beta-D-fructufuranoside fructohydrolase, or a combination thereof.

According to an aspect of the invention, there is preferably disclosed a pharmaceutical composition for improving digestion and/or treating symptoms associated with gastrointestinal complications. The pharmaceutical composition may preferably, but need not necessarily, additionally comprise therapeutically effective amounts of cannabis in any of the following form cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or a combination thereof.

According to an aspect of the invention, there is preferably disclosed a pharmaceutical composition for improving digestion and/or treating symptoms associated with gastrointestinal complications. The pharmaceutical composition may preferably, but need not necessarily, comprise a pharmaceutically acceptable excipient selected from any of the group consisting of: hydroxypropylcellulose, starch, silicon dioxide, gelatin, magnesium stearate, and/or microcrystalline cellulose.

According to an aspect of the invention, there is preferably disclosed a pharmaceutical composition for improving digestion and/or treating symptoms associated with gastrointestinal complications. The pharmaceutical composition may preferably, but need not necessarily, comprise the form of a tablet, caplet, capsule, dermal patch, oil drops, powder or a suspension.

Other advantages, features and characteristics of the present invention, as well as methods of use and applications of the related elements of the pharmaceutical composition and formulation will become more apparent upon consideration of the following detailed description and the appended claims, the latter of which are briefly described hereinbelow.

### DETAILED DESCRIPTION OF THE PREFERRED EMBODIMENTS

The description that follows, and the embodiments described therein, is provided by way of illustration of an example, or examples, of particular embodiments of the principles and aspects of the present invention. These examples are provided for the purposes of explanation, and not of limitation, of those principles and of the invention.

It should also be appreciated that the present invention can be implemented in numerous ways, including as a use of the pharmaceutical composition or a method for improving digestion and/or treating symptoms associated with gastrointestinal complications. In this specification, these implementations, or any other form that the invention may take, may be referred to as uses or methods. In general, the order of the steps of the disclosed methods may be altered within the scope of the invention.

In this disclosure, a number of terms are used. The following definitions of such terms are provided.

As used herein, a person skilled in the relevant art may generally understand the term “comprising” to generally mean the presence of the stated features, integers, steps, or components as referred to in the claims, but that it does not preclude the presence or addition of one or more other features, integers, steps, components or groups thereof.

As used herein, persons skilled in the relevant art will understand that the “gastrointestinal tract” is an organ system within humans and other animals which takes in food, digests it to extract and absorb energy and nutrients, and expels the remaining waste as feces. The mouth, esophagus, stomach, and intestines are components of the gastrointestinal tract.

Persons skilled in the art will understand that the human digestive system consists of the gastrointestinal tract plus the accessory organs of digestion (i.e., tongue, salivary glands, pancreas, liver, and gallbladder). The process of digestion has many stages, the first of which starts in the mouth. As used herein, the term “digestion” means the breakdown of food into progressively smaller components, until they can be absorbed and assimilated into the body. Chewing, in which food is mixed with saliva begins the process of digestion. This produces a bolus which can be swallowed via the esophagus and into the stomach where it is mixed with various enzymes produced by the pancreas. Saliva also contains a catalytic enzyme called amylase which starts to act on food in the mouth. Lingual lipase is another digestive enzyme that is secreted by lingual papillae on the tongue and also from serous glands in the main salivary glands. Gastric juice in the stomach is essential for continuing digestion along with the production of mucus in the stomach. Most of the digestion of food occurs in the small intestine in the gastrointestinal tract. In general, enzymes within the human digestive system (or “digestive enzymes”) are a group of enzymes that aid in the digestion of food by breaking down polymeric macromolecules into their smaller building blocks, in order to facilitate their absorption into the body. The main sites of digestion are the oral cavity, the stomach and the small intestine. Digestive enzymes are secreted by different exocrine glands, including salivary glands, secretory cells in the stomach, secretory cells in the pancreas and secretory glands in the small intestine. Persons skilled in the art will understand that patients can experience various digestive disorders including but not limited to heartburn, gastro-esophageal reflux disease, inflammatory bowel disease and/or irritable bowel syndrome, including symptoms associated with such disorders (e.g., bloating, diarrhea, gas, stomach pain, and/or stomach cramps).

As used herein, a person skilled in the relevant art may generally understand the term “treatment” to generally refer to an approach for obtaining beneficial or desired results. Beneficial or desired results can include, but are not limited to, prevention or prophylaxis, alleviation or amelioration of one or more symptoms or conditions, diminishment of the extent of a disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a person skilled in the relevant art may generally understand the term “therapeutically effective amount” to be an amount sufficient to effect treatment when administered to a subject in need of treatment. In the case of the embodiments of the present invention, a therapeutically effective amount can include, but is not limited to, an amount for improving digestion and/or treating symptoms associated with gastrointestinal complications.

It will be understood by a person skilled in the relevant art that the compositions of the present invention can be formulated into pharmaceutical compositions for administration in a manner customary for administration of such materials using standard pharmaceutical formulation chemistries and methodologies, all of which are readily available to a person skilled in the relevant art. It will also be understood by a person skilled in the relevant art that such pharmaceutical compositions may include one or more excipients, carriers, stabilizers or other pharmaceutically inactive compounds, such as, but not limited to, wetting or emulsifying agents, pH buffering substances, hydroxypropylcellulose, starch, silicon dioxide, gelatin, magnesium stearate, microcrystalline cellulose and the like. Pharmaceutically acceptable salts can also be included therein. A thorough discussion of pharmaceutically acceptable excipients, vehicles and auxiliary substances is available in Remington's. Pharmaceutical Sciences (Mack Pub. Co. N.J. 1991). Such pharmaceutical compositions can be prepared as oral or transdermal preparations. The therapeutically effective doses may vary according to body weight and the timing and duration of administration will be determined by specific clinical research protocols.

It will be understood by a person skilled in the relevant art that the term “dose” refers to the measured quantity of an agent, preferably a therapeutic agent, to be taken at one time to have a desired therapeutic effect(s). Preferably, “dose” as used herein means a specified quantity of a pharmaceutical or therapeutic agent provided in one or more administration. It will be further understood that a “dosage unit” or “dosage form” as used herein means a form in which the active agent is provided. It will be understood that any known dosage form may be employed with the present invention. These may include, solid dosage forms, liquid dosage forms, gel dosage forms, etc. The term “effective dose” or “effective dosage” is defined as an amount sufficient to achieve or at least partially achieve the desired effect. The term “therapeutically effective dose” is defined as an amount sufficient to cure or at least partially arrest the disease and/or one or more associated symptoms in a patient already suffering from the disease.

It will be understood by a person skilled in the relevant art that the term “administering” means providing a therapeutically active agent or composition to a subject, and includes, but is not limited to, administering by a medical professional and self-administering.

It will be understood by a person skilled in the relevant art that a “pharmaceutical agent” or “therapeutic agent” as used herein means a substance that provides a therapeutic effect when administered to a subject. “Pharmaceutical composition” means a mixture of substances suitable for administering to an individual that includes one or more pharmaceutical or therapeutically effective agents. The terms “active pharmaceutical ingredient” shall be understood to refer to a substance in a pharmaceutical composition that provides a desired effect.

Improving digestion and/or treating the symptoms associated with gastrointestinal complications in accordance with the present invention and as hereinafter defined for the purposes of this invention is preferably directed to facilitating the breakdown of larger macromolecules into smaller building blocks and/or relieving symptoms associated with gastrointestinal complications such as bloating, diarrhea, gas, stomach pain and/or stomach cramps. In a preferred embodiment of the present invention, an agent or agents which can improve digestion and/or treating the symptoms associated with gastrointestinal complications is recommended. Since no single active ingredient is presently capable of improving digestion and/or treating the symptoms associated with gastrointestinal complications, a pharmaceutical composition such as is described in the present invention is recommended.

Preferred embodiments of the present invention improve digestion and/or treat symptoms associated with gastrointestinal complications.

In preferable embodiments of the pharmaceutical composition, the combination of cannabis and certain enzymes results in unexpected phenomena. The symptoms associated with the chronic use of cannabis on digestion do not occur.

Preferably, the pharmaceutical compositions of the present invention may be provided with different active ingredients, different strengths and/or different formulations. Preferably, the pharmaceutical composition of the present invention comprises a therapeutically effective amount of cannabis. A person skilled in the relevant art would understand the term “cannabis” to refer to a genus of flowering plants in the family Cannabaceae which produce a group of chemicals called cannabinoids that produce physiological effects when administered to a patient. Persons skilled in the art will also readily appreciate that a cannabinoid is one of a class of diverse chemical compounds that acts on cannabinoid receptors (e.g., cannabinoid receptor type 1, cannabinoid receptor type 2) in cells that alter neurotransmitter release in the brain. In accordance with one or more preferred embodiments of the invention, the pharmaceutical composition may comprise cannabis-derived cannabinoids selected from the group consisting of: cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof. In accordance with an aspect of the present invention, compositions comprising a therapeutically effective amount of cannabis may preferably serve to improve digestion and/or treat symptoms associated with gastrointestinal complications. Preferred embodiments of the pharmaceutical composition may provide higher levels of THC (tetrahydrocannabinol) as it binds the CB (cannabinoid) receptors to help ameliorate the effects in bowel disease. In accordance with some embodiments of the present invention, cannabis may be present in the composition in a particulate form with at least 50% by wt of the particles ranging in size from about 50 micron to about 2000 micron and wherein the particles comprise a cannabinoid, cannabinoid derivative, a terpene or a mixture thereof in a range of about 1 mg to about 30 mg of cannabinoid (CBD or THC) and most preferably from about 2.5 mg to about 10 mg per dose.

Persons of skill in the relevant art may understand that in addition to the cannabis portion of the pharmaceutical composition, certain enzymes may also break down starches and proteins that in turn ease digestion, which may help alleviate some of the gastrointestinal symptoms patients may experience. Accordingly, the pharmaceutical composition of the present invention may also comprise a therapeutically effective amount of one or more enzymes, including but not limited to: 1,4-alpha-D-Glucan glucohydrolase; 4-alpha-D-Glucan glucanohydrolase; Beta-D-galactoside galactohydrolase; Malt diastase; Protease; Triacylglycerol lipase; Beta-D-fructufuranoside fructohydrolase; and combinations thereof

In a preferred embodiment, the pharmaceutical composition of the present invention comprises a therapeutically effective amount of 1,4-alpha-D-Glucan glucohydrolase. Persons skilled in the relevant art would understand the term “1,4-alpha-D-Glucan glucohydrolase” to be an enzyme that catalyses the hydrolysis of alpha D-glucose at the brush border of the small intestine facilitating the breakdown of large sugars into smaller easy to absorb polymers. (See: Avery's Diseases of the Newborn (9th Edition), 2012) In accordance with an aspect of the present invention, compositions comprising a therapeutically effective amount of 1,4-alpha-D-Glucan glucohydrolase may preferably serve to improve digestion and/or treat symptoms associated with gastrointestinal complications. In accordance with some embodiments of the present invention, 1,4-alpha-D-Glucan glucohydrolase may be present in the composition from about 1 mg to about 1000 mg per dose and most preferably from about 5 mg to about 15 mg per dose.

In a preferred embodiment, the pharmaceutical composition of the present invention comprises a therapeutically effective amount of 4-alpha-D-Glucan glucanohydrolase. Persons skilled in the relevant art would understand the term “4-alpha-D-Glucan glucanohydrolase” to be an enzyme that catalyses the hydrolysis of alpha D-glucose at the brush border of the small intestine facilitating the breakdown of large sugars into smaller easy to absorb polymers. (See: Avery's Diseases of the Newborn (9th Edition), 2012) In accordance with an aspect of the present invention, compositions comprising a therapeutically effective amount of 4-alpha-D-Glucan glucanohydrolase may preferably serve improve digestion and/or treat symptoms associated with gastrointestinal complications. In accordance with some embodiments of the present invention, 4-alpha-D-Glucan glucanohydrolase may be present in the composition from about 1 mg to about 1000 mg per dose and most preferably from about 100 mg to about 150 mg per dose.

In a preferred embodiment, the pharmaceutical composition of the present invention comprises a therapeutically effective amount of Beta-D-galactoside galactohydrolase. Persons skilled in the relevant art would understand the term “Beta-D-galactoside galactohydrolase” to be a digestive enzyme that cooperates with other digestive enzymes to breakdown carbohydrates, fats and protein. In accordance with some embodiments of the present invention, Beta-D-galactoside galactohydrolase may be present in the composition from about 1 mg to about 1000 mg per dose and most preferably from about 25 mg to 75 mg per dose.

In a preferred embodiment, the pharmaceutical composition of the present invention comprises a therapeutically effective amount of Malt diastase. Persons skilled in the relevant art would understand the term “Malt diastase” to be a digestive enzyme that cooperates with other digestive enzymes to breakdown carbohydrates, fats and protein. In accordance with an aspect of the present invention, compositions comprising a therapeutically effective amount of Malt diastase may preferably serve to improve digestion and/or treat symptoms associated with gastrointestinal complications. In accordance with some embodiments of the present invention, Malt diastase may be present in the composition from about 1 mg to about 1000 mg per dose and most preferably from about 10 mg to about 30 mg per dose.

In a preferred embodiment, the pharmaceutical composition of the present invention comprises a therapeutically effective amount of Protease. Persons skilled in the relevant art would understand the term “Protease” to be a digestive enzyme that cooperates with other digestive enzymes to breakdown carbohydrates, fats and protein. In accordance with an aspect of the present invention, compositions comprising a therapeutically effective amount of Protease may preferably serve to improve digestion and/or treat symptoms associated with gastrointestinal complications. In accordance with some embodiments of the present invention, Protease may be present in the composition from about 1 mg to about 1000 mg per dose and most preferably from about 100 mg to about 150 mg per dose.

In a preferred embodiment, the pharmaceutical composition of the present invention comprises a therapeutically effective amount of Triacylglycerol lipase. A person skilled in the relevant art would understand the term “Triacylglycerol lipase” to be a digestive enzyme that cooperates with other digestive enzymes to breakdown carbohydrates, fats and protein. In accordance with an aspect of the present invention, compositions comprising a therapeutically effective amount of Triacylglycerol lipase may preferably serve to improve digestion and/or treat symptoms associated with gastrointestinal complications. In accordance with some embodiments of the present invention, Triacylglycerol lipase may be present in the composition from about 1 mg to about 1000 mg per dose and most preferably from about 10 mg to about 30 mg per dose.

In a preferred embodiment, the pharmaceutical composition of the present invention comprises a therapeutically effective amount of Beta-D-fructufuranoside fructohydrolase. A person skilled in the relevant art would understand that “Beta-D-fructufuranoside fructohydrolase” to be a digestive enzyme that cooperates with other digestive enzymes to breakdown carbohydrates, fats and protein. In accordance with an aspect of the present invention, compositions comprising a therapeutically effective amount of Beta-D-fructufuranoside fructohydrolase may preferably serve to improve digestion and/or treat symptoms associated with gastrointestinal complications. In accordance with some embodiments of the present invention, Beta-D-fructufuranoside fructohydrolase may be present in the composition from about 1 mg to 1000 mg per dose and most preferably from about 5 mg to 20 mg per dose.

In a preferred embodiment of the present invention, the combination of cannabis with digestive enzymes provides advantageous outcomes as the pharmaceutical composition retains the therapeutic benefits of cannabis on relief of gastrointestinal disorders while minimizing its potential adverse effects and facilitating more efficient digestion and improvement with regards to gastrointestinal symptoms a patient may be experiencing.

The pharmaceutical composition may preferably, but need not necessarily, be administered prior to each meal. In preferable, embodiments, a dose is taken multiple times daily prior to each meal when a patient is experiencing digestive upset and/or symptoms due to gastrointestinal complications. In an alternate embodiment, the pharmaceutical composition may be administered following a meal. In a preferred embodiment, a dosing regimen is continued until the digestive upset and/or symptoms due to gastrointestinal complications has abated. In some embodiments, the composition of the present invention may be administered once daily to prevent digestive upset and/or symptoms due to gastrointestinal complications.

In a preferred embodiment, the pharmaceutical composition of the present invention is used as a nutritional supplement to help patients who suffer from indigestion, bloating, inflammatory bowel disease or irritable bower syndrome. The pharmaceutical composition of the present invention will preferably minimize the feeling of nausea, indigestion, dyspepsia, bloating, flatulence, stomach aches or cramps, gas, diarrhea, functional bowel disorders and decrease flares of inflammatory bowel disease in mild to moderate cases. The pharmaceutical composition of the present invention is preferably formulated in multiple strengths to treat the symptoms associated with the above disorders. The pharmaceutical composition of the present invention is preferably formulated to use the minimal therapeutically effective doses of the active ingredients to maximize absorption in the gastrointestinal track (e.g., maximize bioavailability) and minimize side effects. In accordance with a preferred embodiment of the present invention, the pharmaceutical composition allows for a reduction in the dose of cannabis required to achieve the same therapeutic effect compared to cannabis administered to a patient in a traditional dosage form (e.g., smoking or inhalation).

In accordance with a preferred embodiment of the present invention, the administration of lower doses of cannabis (compared to cannabis administered in a traditional dosage form, for example, smoking or inhalation) combined with the enzymes disclosed herein is advantageous as it provides a therapeutic effect on improving digestion and/or treating symptoms associated with gastrointestinal complications and a reduction in cravings for further cannabis that chronic cannabis users may experience.

In accordance with a preferred embodiment of the present invention, the combination of cannabis and/or enzymes disclosed herein is advantageous as it may provide an unexpected synergistic therapeutic effect on improving digestion and/or treating symptoms associated with gastrointestinal complications.

In accordance with a preferred embodiment of the present invention, the combination of cannabis and/or enzymes disclosed herein is advantageous as it may unexpectedly allow for the use of a lower dose of cannabis to achieve a similar therapeutic effect when compared to cannabis administered to a patient in a traditional dosage form (e.g., smoking or inhalation).

In accordance with a preferred embodiment of the present invention, the combination of cannabis and/or enzymes disclosed herein is advantageous as it may allow for the use of a lower dose of cannabis to reduce the severity and/or number of potential cannabis-related adverse effects.

The pharmaceutical composition of the present invention is a formulation comprising cannabis in addition to various digestive enzymes, either used as a medicine prescribed by a physician, a health care practitioner, or an over-the-counter product available in pharmacies, marijuana dispensaries, and mass food stores and will preferably help patients who suffer from gastrointestinal disorders and will improve symptom relief for nausea, bloating, diarrhea and inflammatory bowel disease.

In accordance with a preferred embodiment of the present invention, the pharmaceutical composition comprising cannabis and digestive enzymes may be administered prior to each meal or shortly after a meal. The pharmaceutical composition of the present invention delivers high concentrations of several digestive enzymes including 1,4-alpha-D-Glucan glucohydrolase, 4-alpha-D-Glucan glucanohydrolase, Beta-D-galactoside galactohydrolase, Malt diastase, Protease, Triacylglycerol lipase, Beta-D-fructufuranoside fructohydrolase and therapeutically effective concentrations of cannabis in any form without causing increased side effects. The combination of the foregoing enzymes and cannabis in a single formulation for improving digestion and/or treating symptoms associated with gastrointestinal complications is unique and has not been previously described.

### EXAMPLE 1

The following example sets out a preferred formulation of the pharmaceutical composition in accordance with an aspect of the present invention for improving digestion and/or treating symptoms associated with gastrointestinal complications.

| Medicinal Ingredients | Quantity | |
|---|---|---|
| 1,4-alpha-D-Glucan glucohydrolase | 10.62 | FCC AGU |
| | 10.62 | mg |
| 4-alpha-D-Glucan glucanohydrolase | 133.65 | mg |
| | 5940.0 | FCC DU |
| Beta-D-galactoside galactohydrolase | 50.0 | FCC ALU |
| | 50.0 | mg |
| Malt diastase | 2.0 | FCC DP |
| | 20.0 | mg |
| Protease | 133.65 | mg |
| | 14850.0 | FCC HUT |
| Triacylglycerol lipase | 23.76 | mg |
| | 297.0 | FCC LU |
| beta-D-fructofuranoside | 10.0 | FCC INVU |
| fructohydrolase | 10.0 | mg |
| Cannabis (THC) | 2.5 | mg |

In preferred embodiments of the invention, the formulation is allergen free (e.g., egg products, wheat (gluten) and dairy (lactose)). In addition, all compounds in the formulation are preferably gluten free.

The above description is meant to be exemplary only, and one skilled in the art will recognize that changes may be made to the embodiments described without departing from the scope of the invention disclosed. Modifications which fall within the scope of the scope of the present invention will be apparent to those skilled in the art, in light of a review of this disclosure, and such modifications are intended to fall within the appended claims.

This concludes the description of presently preferred embodiments of the invention. The foregoing description has been presented for the purpose of illustration and is not intended to be exhaustive or to limit the invention to the precise form disclosed. Other modification, variations and alterations are possible in light of the above teaching and will be apparent to those skilled in the art, and may be used in the design and manufacture of other embodiments according to the present invention without departing from the spirit and scope of the invention. It is intended the scope of the invention be limited not by this description but only by the claims forming a part hereof.

What is claimed is:

1. A pharmaceutical composition for improving digestion and/or treating the symptoms associated with gastrointestinal complications in a patient comprising a therapeutically effective amount of cannabis, an enzyme and pharmaceutically acceptable excipients.

2. The pharmaceutical composition of claim 1, wherein the cannabis is selected from the group consisting of: cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), a naphthoylindole, a phenylacetylindole, a benzoylindole, a cyclohexylphenole, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), the pharmaceutical agent is CBD, THC or combinations thereof

3. The pharmaceutical composition of claim 2, wherein the enzyme is selected from the group consisting of: 1,4-alpha-D-Glucan glucohydrolase; 4-alpha-D-Glucan glucanohydrolase; Beta-D-galactoside galactohydrolase; Malt diastase; Protease; Triacylglycerol lipase; Beta-D-fructufuranoside fructohydrolase; and combinations thereof.

4. The pharmaceutical composition of claim 3 comprising: 1,4-alpha-D-Glucan glucohydrolase from about 1 mg to about 1000 mg per dose; 4-alpha-D-Glucan glucanohydrolase from about 1 mg to about 1000 mg per dose; Beta-D-galactoside galactohydrolase from about 1 mg to about 1000 mg per dose; Malt diastase from about 1 mg to about 1000 mg per dose; Protease from about 1 mg to about 1000 mg per dose; Triacylglycerol lipase from about 1 mg to about 1000 mg per dose; and/or Beta-D-fructufuranoside fructohydrolase from about 1 mg to about 1000 mg per dose.

5. The pharmaceutical composition of claim 3 comprising: 1,4-alpha-D-Glucan glucohydrolase from about 5 mg to about 15 mg per dose; 4-alpha-D-Glucan glucanohydrolase from about 100 mg to about 150 mg per dose; Beta-D-galactoside galactohydrolase from about 25 mg to about 75 mg per dose; Malt diastase from about 10 mg to about 30 mg per dose; Protease from about 100 mg to about 150 mg per dose; Triacylglycerol lipase from about 10 mg to about 30 mg per dose; and/or Beta-D-fructufuranoside fructohydrolase from about 5 mg to about 20 mg per dose.

6. The pharmaceutical composition of claim 5, wherein the cannabis comprises from about 1 mg to about 30 mg of cannabinoid per dose.

7. The pharmaceutical composition of claim 5, wherein the cannabis comprises from about 2.5 mg to about 10 mg of cannabinoid per dose.

8. Use of a pharmaceutical composition comprising a therapeutically effective amount of cannabis, an enzyme and pharmaceutically acceptable excipients for improving digestion and/or treating the symptoms associated with gastrointestinal complications in a patient.

9. A method for improving digestion and/or treating the symptoms associated with gastrointestinal complications by administering to the patient, in one or more doses, a pharmaceutical composition comprising a therapeutically effective amount of cannabis, an enzyme and pharmaceutically acceptable excipients.
